(19) United States
(12) Patent Application Publication
(10) Pub. No.: US 2008/0060639 A1
(43) Pub. Date: Mar. 13, 2008

(54) AIR CHAMBER FOR APPLYING AEROSOL DRUGS WITH GEOMETRIC VALVE

(76) Inventor: CESAR EDUARDO MARQUES Da SILVA, Sao Paulo/SP (BR)

Correspondence Address: MCKEE, VOORHEES & SEASE, P.L.C., 801 GRAND AVENUE, SUITE 3200, DES MOINES, IA, 50309-2721, US

(21) Appl. No.: 11/755,267

(22) Filed: May 30, 2007

(30) Foreign Application Priority Data
Sep. 13, 2006 (BR) PI 0603695-3

(51) Int. Cl. A61M 11/00

(52) U.S. Cl. 128/200.14

(57) ABSTRACT

“AIR CHAMBER FOR APPLYING AEROSOL DRUGS WITH GEOMETRIC VALVE” particularly an air chamber (A) indicated for aerosol drug application that stands out for having a geometric valve (1) of variable cross-section, optimized constructive layout consisting of two parts (2), forming an aerosol deceleration sub-chamber (3) and another mix sub-chamber (4), in addition to a sealed compartment (C) for receiving the aerosol container (5) run by a driving shield (6) coupled to the main assembly.

### CROSS-REFERENCE TO RELATED APPLICATIONS

This application claims priority as required by 35 U.S.C. § 119(b) and 37 CFR 1.55(a) of Brazilian Application No. PI 0603695-3 filed Sep. 13, 2006, which application is hereby incorporated by reference in its entirety.

### BRIEF SUMMARY

This patent application is about the invention of an “AIR CHAMBER FOR APPLYING AEROSOL DRUGS WITH GEOMETRIC VALVE”, particularly an air chamber designed for applying aerosol-type drugs by breathing that stands out for being fitted with a geometric valve in its mid-body, of reduced volume that optimizes portability, where said valve has the role of subdividing the inner part into two sub-chambers so as to decelerate aerosol particles by increasing breathable particle mass inside the air chamber.

### THEORETICAL BASIS

Every aerosol drug is designed for applying to the respiratory tact under the form of gas mixed with air to be then laid in various lung regions. Laying can occur by inertial impact; gravitational sedimentation and spreading over the lung surface.

Drug amount that actually reaches lungs depends on aerosol particle physical properties; factors optimizing gas/air mix and respiratory tract and lung mechanics.

Under ideal circumstances it is possible to reach deposit rates of this kind of drug (aerosol) higher than 50%. Since factors affecting deposit are very complex, such drivers are incorporated into the application system design, taking into consideration the cost/benefit ration; behavioral factors and patient's profile.

The Air Chamber implies improvements that provide aerosol or gas retention, injected from a pressurized container (pMDI—pressurized Meter Dose Inhalers), inside the chamber for sufficiently long time, in order to produce the greatest possible mass of breathable particles.

Gas particles of aerosol drugs available on the market reach speed ranges from 10 to 100 m/s, therefore not being typically breathable. When directly sprayed into the mouth cavity a large mass of these particles is lost by evaporation; by retention in the mouth cavity; tongue; pharynx and respiratory tract in general.

### THE STATE OF TECHNIQUE

In the current STATE OF TECHNIQUE, there are four most utilized techniques for applying therapeutic aerosols: jet sprayers; ultrasonic sprayers, pMDI's (Pressurized Meter Dose Inhalers) known as “pump” and DPI's (Dry Powder Inhalers) the “powder”.

From cost×benefit point of view, portability, deposit effectiveness, convenience or easiness of application pMDI's and DPI's are the recommended techniques by specialists as a priority option on the part of physicians for treating respiratory diseases.

In the case of pMDI's the use of Air Chambers with valves is further recommended, whenever the patient does not present sufficient hand movement coordination and breathing and when deposit in the pharynx (steroid inhalation) is a reason for concern. In relation to above-commented breathing, aerosol application, quite in advance, during inhalation places the drug in the frontal part of respiration producing a deeper penetration. In contrast, late aerosol application in inhalation increases the drug amount in the final respiration flow favoring deposit in the lung regions that receive air ultimately.

In geriatric and pediatric populations the use of air chambers with mask is often recommended. The consensus of specialists further suggests to consider the patient's preferences in selecting the application device: determining behavioral aspects of such preferences are particularly important in adolescence and late childhood stages.

Currently there is a wide range of devices on the market, sometimes erroneously labeled as Air Chamber. Often, the term spacer, is used as an air chamber synonym.

In systems for orally applied pressurized aerosol in respiratory therapy, it is important to distinguish that any device that keeps that drug away from the mouth cavity may be referred to as spacer.

### DEFICIENCIES OF CONVENTIONAL AIR CHAMBERS

Although there is consensus in relation to benefits of air chambers in aerosol application, the debate in relation to the ideal size of air chambers remains. Scientific evidence supporting the use of large or small devices is inconsistent, as far as treatment effectiveness is concerned.

On the other hand, the consensus of specialists is that regardless of the chamber volume, which may range from 145 ml to 700 ml, in existing devices on the market, the size is one of the main disadvantages and consequently makes acceptance of the part of potential users difficult.

Furthermore, the shape of air chambers is generally cylindrical and seldom unsuitable for being carried in pockets of common clothing articles, in addition to the fact of being forcefully used in the upright position.

Although there are small volume devices and a more convenient shape for carrying, they meet more spacer than air chamber definition, in which aerosol jet is applied at high speed into the mouth cavity, practically with the same usage disadvantages as pMDI with no air chamber.

### THE PURPOSE OF THE INVENTION

Therefore, the purpose of this innovation is to provide a product that broadly meets accepted requirements by the scientific community, with significant advantages in the portability aspect and acceptance on the part of young and adult patients, which should meet requirements listed below;
producing maximum breathable particle mass;
aerosol generation and particle size to be independent of inhalation speed;
particle size to be independent of ambient humidity;
non-obstructive and fitting in the pocket;
affordable cost;
containing a device that stimulates slow inhalation;
signaling at 0.45 l/min, a too high rate in pMDI;
quick administration of prescribed dose;
dissociation from aerosol generation and inhalation;
that exhalation prior to inhalation will not disperse nor moisten the mix;
deposit maximization in Lower Respiratory Tract (LRT) by particle selectivity and deposit minimization in Upper Respiratory Tract (URT).
Minimization of dead space, that is, not being used;
low air passage-resistant inhalation and exhalation valve;
Appropriate masks for breastfeeding women, children and adults;
simplified operation to any kind of user.

### THE INVENTION

At the upper end of the performance spectrum of the aerosol application devices are cylindrical valve-fitted Air Charmers with a mask and a the lower end are spacers that merely keep the aerosol jet away from the mouth cavity with no improvement in the breathable particle mass though they offer the advantage as far as size and portability are concerned.

The air chamber with a geometric valve matches the small volume that optimizes portability to a geometric valve system that decelerates aerosol particles increasing the breathable particle mass inside the chamber.

The compact, discreet configuration of the air chamber with a geometric valve and top efficiency in smaller, more breathable particle generation, facilitates adhesion to using air chambers by adolescent and adult patients in general.

Resistance to using conventional valve-fitted air chambers, due to behavioral issues, is highly harmful to asthma treatment.

This new configuration and constructive design, create an air chamber with a smaller number of components and lower production and assemblage costs.

### ADVANTAGES OF THE INVENTION

it does not require a cap for obtaining a high breathable particle mass;
better mixing conditions than similar ones in size and shape;
smaller number of parts (three parts with a cap or two parts with no cap);
lower production and assemblage cost;
ergonomically suitable and more comfortable handhold for pMDI driving, which breaks the upright use paradigm.
it can be used with a coupled mask.

### DESCRIPTION OF FIGURES

The invention will be explained, as follows, with reference to drawing listed below, in illustrative and non-limiting manner:

FIG. 1: View in perspective of the innovated air chamber;

FIG. 2: Exploded view in perspective of the innovated air chamber;

FIG. 3: View in partial sectioned perspective of the innovated air chamber;

FIG. 4: View in partial inverted sectioned perspective of the innovated air chamber;

FIG. 5: Side view in inverted section of the innovated air chamber;

FIG. 6: View in perspective of the innovated air chamber in a constructive variation.

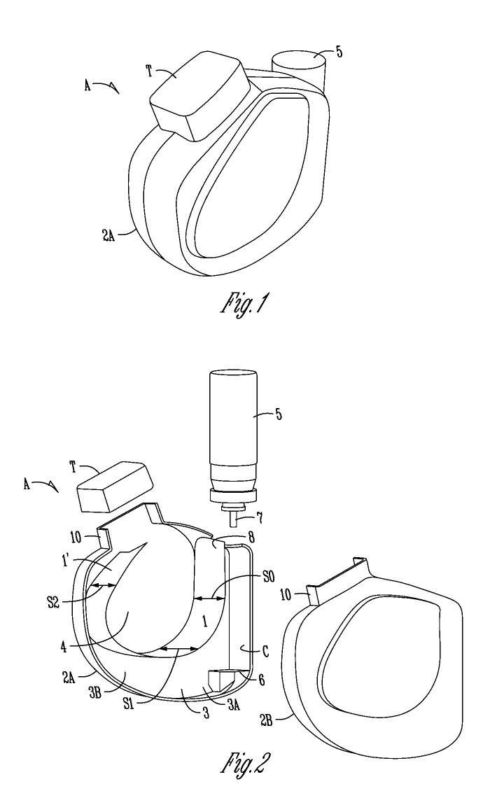

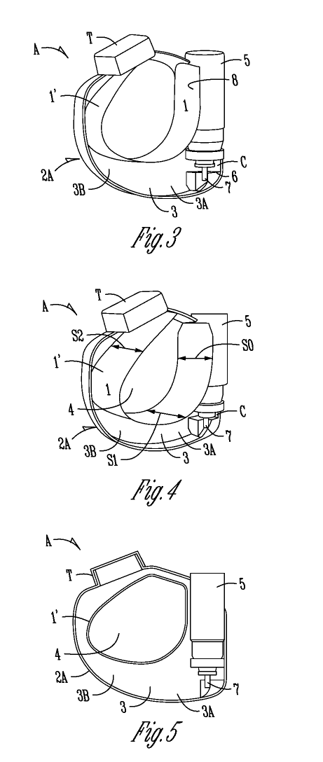

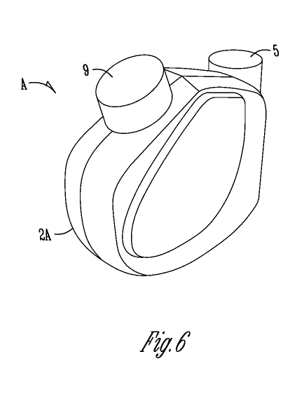

### DETAILED DESCRIPTION

The “AIR CHAMBER FOR APPLYING AEROSOL DRUGS WITH GEOMETRIC VALVE”, the reason for this Utility Model patent application, is indicated for aerosol drug application that stands out for having a geometric valve (1) of variable cross-section (S), optimized constructive layout consisting of two parts (2A and 2B), forming an aerosol deceleration sub-chamber (3) and another mix sub-chamber (4), in addition to a sealed compartment (C) for receiving the aerosol container (5) run by a driving shield (6) coupled to one of the parts (2A) which form the Air Chamber (A).

More specifically, the Air Chamber (A) basically consists of two injected parts (2A and 2B) that are coupled and fitted into each other forming a retention and mix sub-chamber (4), and a second aerosol deceleration sub-chamber (3) in addition to an isolated compartment (C), into which the aerosol container (5) containing the drug is coupled.

Around the upper median portion of the product a semi-spiral (1′) is projected with a variable cross-section (S0; S1 and S2) which is nothing else than the geometric valve (1), bounding the two sub-chambers (3 and 4); more specifically the deceleration chamber (3) and mix chamber (4).

The deceleration chamber (3) has an initial low-pressure zone (3A) near the nozzle (7) of the pMDI and a high-pressure zone (3B) created by the aerosol passage area reduction. In this region the aerosol swirling occurs, which when reducing the speed is forced over the semi-spiral (1′) toward the mixing chamber (4).

In this initial portion (8) of the semi-spiral (1′), there is a slight reduction in the cross-section (S1) in order to allow decelerated aerosol entry into the mixing chamber (4) followed by a differentiated section (S2).

The semi-spiral (1′) geometry, which divides deceleration (3) and mixing (4) chambers is designed to maximize deceleration, so that a negligible amount of aerosol breathable particle mass leaves the mixing chamber (4) without inhalation on part of the patient.

The air chamber (A) can be fitted with a rectangular projection (10) of ergonomically designed outlet for placing the mouth, which is fitted with a cap (T) in order to ensure the best hygiene conditions while not in use.

A small sound sensor (not represented) activated when inhalation speed exceeds 0.45 l/min can be installed in more specific product versions.

In a constructive variation, the air chamber (A) can be fitted with a cylindrical projection (9) designed for coupling the inhalation mask, preferably with in exhalation valve.

1) “AIR CHAMBER FOR APPLYING AEROSOL DRUGS WITH GEOMETRIC VALVE”, characterized by consisting of two injected parts (2 A and 2B) that are coupled and fitted into each other forming a retention and mix sub-chamber (4), and a second aerosol deceleration sub-chamber (3) in addition to an isolated compartment (C), into which the aerosol container (5) containing the drug is coupled; around the upper median portion of the product a semi-spiral (1′) is projected with a variable cross-section (S0; S1 and S2) which is nothing else than the geometric valve (1), bounding the two sub-chambers (3 and 4); the deceleration chamber (3) has an initial low-pressure zone (3A) near the nozzle (7) of the pMDI and a high-pressure zone (3B) created by the aerosol passage area reduction. forced over the semi-spiral (1′) toward the mixing chamber (4); in the initial portion (8) of the semi-spiral (1′) the cross-section has an internal width equal to the Air Chamber (A) bounding the pMDI compartment (C), providing full sealing of the low-pressure zone (3 A); in the initial portion of the semi-spiral (1′), there is a slight reduction in the cross-section (S1) in order to allow decelerated aerosol entry into the mixing chamber (4) followed by a differentiated section (S2); the air chamber (A) can be fitted with a rectangular projection (10) of ergonomically designed outlet for placing the mouth, which is fitted with a cap (T); a small sound sensor (not represented) activated when inhalation speed exceeds 0.45 l/min can be installed in more specific product versions; in a constructive variation, the air chamber (A) can be fitted with a cylindrical projection (9) designed for coupling the inhalation mask, preferably with in exhalation valve.
